(19) United States
(12) Patent Application Publication
(10) Pub. No.: US 2023/0190974 A1
(43) Pub. Date: Jun. 22, 2023

(54) Shoe Sanitizing Assembly

(71) Applicant: Eileen Belfon, East Orange, NJ (US)

(72) Inventor: Eileen Belfon, East Orange, NJ (US)

(21) Appl. No.: 17/553,293

(22) Filed: Dec. 16, 2021

(51) Int. Cl. A61L 2/22

(52) U.S. Cl. CPC A61L 2/22; A61L 2202/14; A61L 2202/15

(57) ABSTRACT

A shoe sanitizing assembly for sanitizing soles of shoes includes a floor mat that is positionable on a floor and the floor mat is stood upon by a user. The floor mat has a pair of foot areas to have a sole of a respective shoe placed in the foot areas. A pressure switch is integrated into the floor mat and the pressure switch is compressed when the user stands on the floor mat. A reservoir unit is coupled to the floor mat and the reservoir unit is aligned with a respective one of four corners of the floor mat. The reservoir unit contains a fluid sanitizer and the reservoir unit is in operational communication with the pressure switch. A sprayer is integrated into the floor mat to spray the fluid sanitizer on the soles of the shoes when the user steps on the floor mat.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sanitizing devices and more particularly pertains to a new sanitizing device for sanitizing soles of shoes. The device includes a pan, a floor mat disposed in the pan and a pair of pads integrated into the floor mat which are each comprised of a fluid permeable material. The device includes a canister that is disposed on the pan, which contains a fluid sanitizer, and a sprayer which is positioned between the pan and the floor mat. The sprayer sprays the fluid sanitizer upwardly through each of the pads to sanitize the sole of shoes when a user stands on the pads.

### (2) Description of Related Art Including Information Disclosed Under 37 CFR 1.97 and 1.98

The prior art relates to sanitizing devices including a shoe cleaner device that includes a floor mat with a brush section and a remotely positioned fluidly reservoir. The prior art discloses a shoe cleaner device that includes a pan which contains a sanitizing fluid and permeable membrane stretched over the pan. The prior art discloses a shoe sanitizing device that comprises a floor may which comprises a plurality of layers and a spraying unit that is disposed between respective layers for spraying a fluid sanitizer. The prior art discloses an antibacterial floor mat that has bio-detection element for detecting bacteria on a sole of a shoe. The prior art discloses a disinfecting floor mat that is infused with an antibacterial solution. The prior art discloses a disinfecting floor mat device that includes a floor mat and a spray can that is disposed on the floor mat for spraying an sanitizing solution on the floor mat.

### BRIEF SUMMARY OF THE INVENTION

An embodiment of the disclosure meets the needs presented above by generally comprising a floor mat that is positionable on a floor and the floor mat is stood upon by a user. The floor mat has a pair of foot areas to have a sole of a respective shoe placed in the foot areas. A pressure switch is integrated into the floor mat and the pressure switch is compressed when the user stands on the floor mat. A reservoir unit is coupled to the floor mat and the reservoir unit is aligned with a respective one of four corners of the floor mat. The reservoir unit contains a fluid sanitizer and the reservoir unit is in operational communication with the pressure switch. A sprayer is integrated into the floor mat to spray the fluid sanitizer on the soles of the shoes when the user steps on the floor mat.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a shoe sanitizing assembly according to an embodiment of the disclosure.

FIG. 2 is a top phantom view of an embodiment of the disclosure.

FIG. 3 is a left side cut-away view of an embodiment of the disclosure.

FIG. 4 is a perspective in-use view of an embodiment of the disclosure.

### DETAILED DESCRIPTION OF THE INVENTION

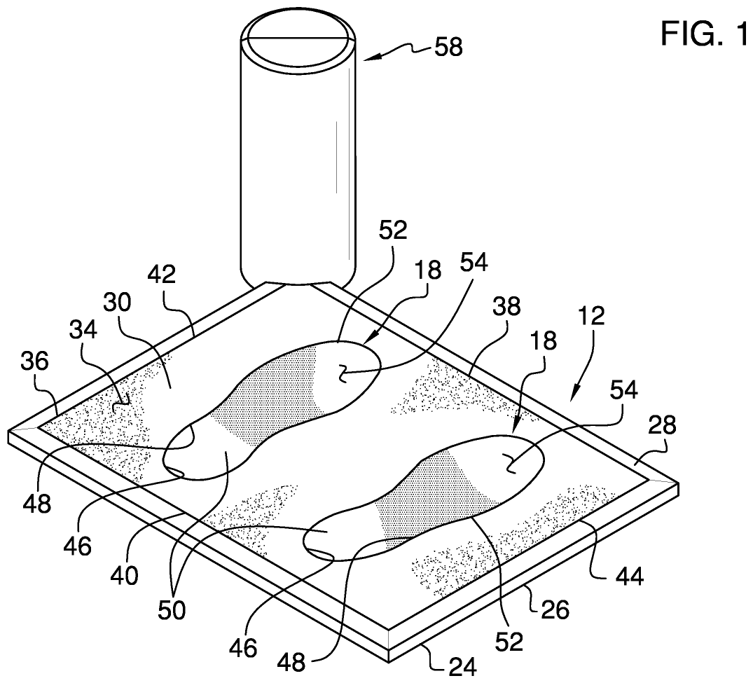

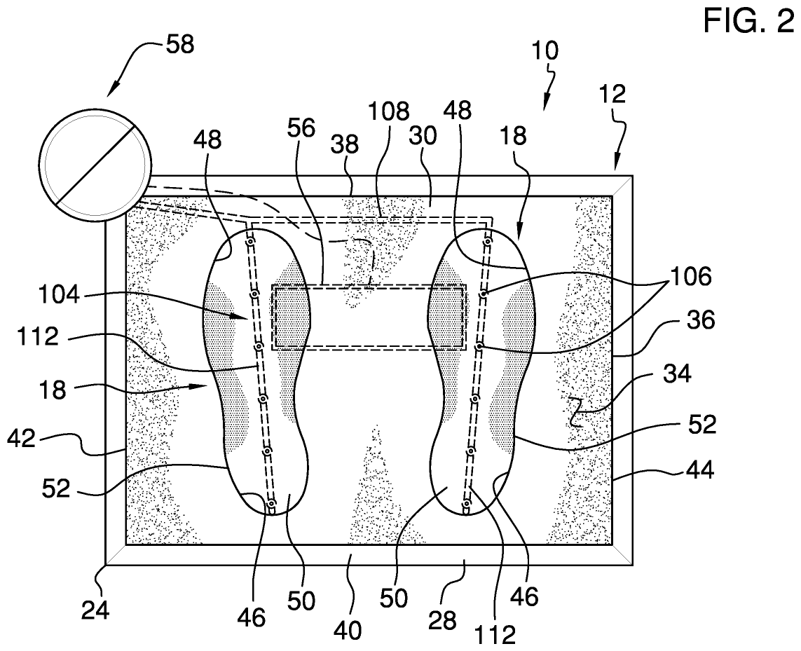

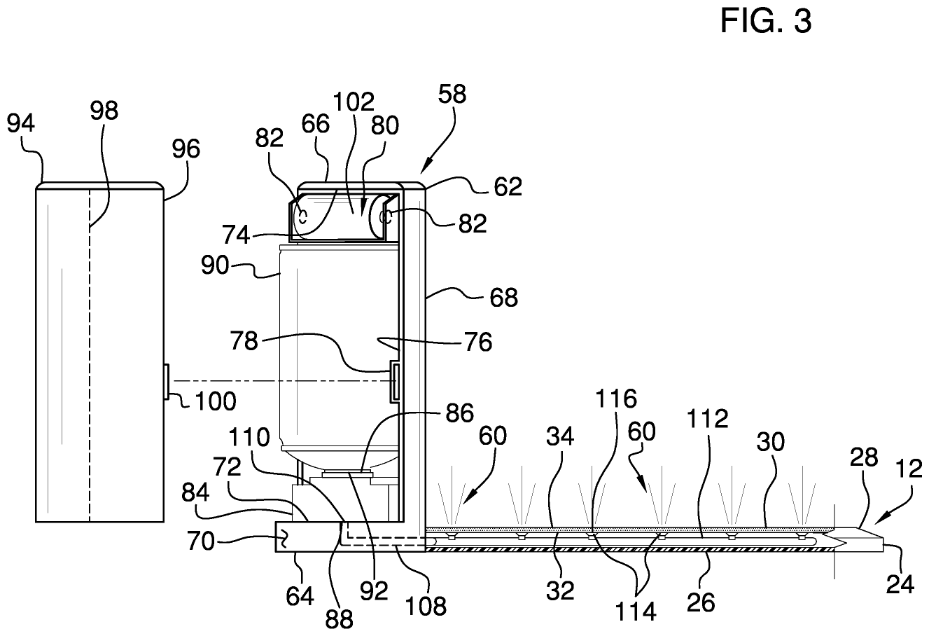

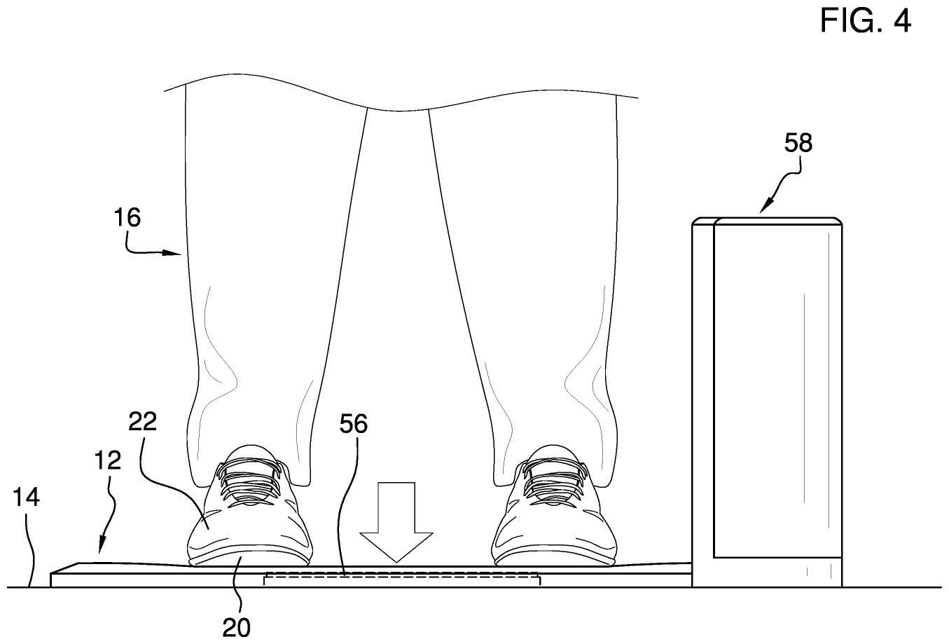

With reference now to the drawings, and in particular to FIGS. 1 through 4 thereof, a new sanitizing device embodying the principles and concepts of an embodiment of the disclosure and generally designated by the reference numeral 10 will be described.

As best illustrated in FIGS. 1 through 4, the shoe sanitizing assembly 10 generally comprises a floor mat 12 that is positionable on a floor 14 such that the floor mat 12 can be stood upon by a user 16. The floor mat 12 has a pair of foot areas 18 each defined on the floor mat 12 such that each of the foot areas 18 can have a sole 20 of a respective shoe 22 placed in the foot areas 18. Additionally, each of the foot areas 18 is comprised of a fluid permeable material. The floor mat 12 comprises a pan 24 that has a bottom wall 26 and a perimeter wall 28 extending upwardly from the bottom wall 26. The pan 24 is comprised of a rigid material and the pan 24 is comprised of a fluid impermeable material. The floor mat 12 includes a panel 30 that has a bottom surface 32, a top surface 34 and a perimeter surface 36 extending between the top surface 34 and the bottom surface 32. The perimeter surface 36 is bonded to the perimeter wall 28 of the pan 24 such that the panel 30 completely fills an area defined by the perimeter wall 28 of the pan 24. The bottom surface 32 is spaced from an upper surface of the bottom wall 26 of the pan 24, and the perimeter surface 36 has a front side 38, a back side 40, a first lateral side 42 and a second lateral side 44.

The panel 30 has a pair of holes 46 each extending through the top surface 34 and the bottom surface 32 such that each of the holes 46 defines a respective one of the foot areas 18. Each of the holes 46 is elongated along an axis extending between the front side 38 and the back side 40 of the perimeter surface 36 such that each of the holes 46 has an ovoid shape. Each of the holes 46 has a bounding edge 48 and the bounding edge 48 of each of the holes 46 undulates along opposing sides of the ovoid shape defined by the holes 46 such that each of the holes 46 has a shape that approximates the shape of the sole 20 of the shoe 22. Each of the holes 46 is positioned on opposite sides of a center of the panel 30.

The floor mat 12 includes a pair of pads 50 that each of the pads 50 has an outer edge 52. Each of the pads 50 is positioned in a respective one of the holes 46 having the outer edge 52 abutting the bounding edge 48 of the respective hole 46. Furthermore, each of the pads 50 completely fills the respective hole 46 such that each of the pads 50 can be stood upon. Each of the pads 50 is comprised of a fluid permeable material and an upper surface 54 of each of the pads 50 is aligned with the top surface 34 of the panel 30.

A pressure switch 56 is integrated into the floor mat 12 and the pressure switch 56 is compressed when the user 16 stands on the floor mat 12. The pressure switch 56 is turned on when the user 16 stands on the floor mat 12 and the pressure switch 56 is positioned between the bottom wall 26 of the pan 24 and the panel 30. The panel 30 presses downwardly on the pressure switch 56 when the user 16 stands on the panel 30, and the pressure switch 56 is elongated to extend between each of the pads 50.

A reservoir unit 58 is coupled to the floor mat 12 and the reservoir unit 58 is aligned with a respective one of four corners of the floor mat 12. The reservoir unit 58 contains a fluid sanitizer 60 and the reservoir unit 58 is in operational communication with the pressure switch 56. The reservoir unit 58 is actuated to release a pre-determined volume of the fluid sanitizer 60 when the pressure switch 56 is turned on. The reservoir unit 58 comprises a housing 62 that has a lower disk 64, an upper disk 66 and a riser 68 extending between the lower disk 64 and the upper disk 66. The lower disk 64 has an outer surface 70 and a topmost surface 72, the upper disk 66 has a lowermost surface 74 and the riser 68 has a lateral edge 76.

The lateral edge 76 has an engagement 78 that is disposed on the lateral edge 76. The outer surface 70 of the lower disk 64 is coupled to the perimeter wall 28 of the pan 24 of the floor mat 12. Additionally, the upper disk 66 has a power supply compartment 80 that is disposed on the lowermost surface 74. The power supply compartment 80 has a set of contacts 82 that is integrated into the power supply compartment 80, and each of the set of contacts 82 is comprised of an electrically conductive material.

The reservoir unit 58 includes a pump 84 that is disposed on the topmost surface 72 of the lower disk 64. The pump 84 has an intake 86 and an exhaust 88, and the pump 84 is electrically coupled to the pressure switch 56. The pump 84 is turned on when the pressure switch 56 is turned on and the pump 84 is electrically coupled to the pressure switch 56. The pump 84 may comprise an electric fluid pump or the like. The reservoir unit 58 includes a canister 90 which contains the fluid sanitizer 60. The canister 90 has an outlet 92 and the canister 90 is removably positionable in the housing 62 having the outlet 92 fluidly engaging the intake 86 of the pump 84. The pump 84 urges a pre-determined volume of the fluid sanitizer 60 outwardly from the canister 90 when the pump 84 is turned on.

The reservoir unit 58 includes a cover 94 that has a first lateral edge 96 and a second lateral edge 98, and the cover 94 is concavely arcuate between the first lateral edge 96 and the second lateral edge 98. The first lateral edge 96 has an engaging element 100 that is disposed on the first lateral edge 96. The cover 94 is positionable in the housing 62 having the cover 94 extending between the upper disk 66 and the lower disk 64. The engaging element 100 releasably engages the engagement 78 on the lateral edge 76 of the riser 68 when the cover 94 is positioned in the housing 62. A power supply 102 is provided, the power supply 102 is insertable into the power supply compartment 80 and the power supply 102 comprises at least one battery. The power supply 102 is placed in electrical communication with each of set of contacts 82 in the power supply compartment 80 when the power supply 102 is inserted into the power supply compartment 80.

A sprayer 104 is integrated into the floor mat 12 and the sprayer 104 is in fluid communication with the reservoir unit 58 such that the sprayer 104 receives the fluid sanitizer 60 from the reservoir unit 58. The sprayer 104 has a plurality of spraying elements 106 that is each aligned with a respective one of the foot areas 18 in the floor mat 12. In this way each of the spraying elements 106 can spray the fluid sanitizer 60 on the sole 20 of the shoe 22 when the user 16 steps on the floor mat 12. The sprayer 104 comprises a primary conduit 108 that is positioned between the bottom surface 32 of the panel 30 and the upper surface of the bottom wall 26 of the pan 24. The primary conduit 108 has first end 110 that is fluidly coupled to the exhaust 88 of the pump 84 such that the primary conduit 108 receives the fluid sanitizer 60 from the pump 84.

The sprayer 104 includes a pair of secondary conduits 112 that is each fluidly coupled to the primary conduit 108 such that each of the secondary conduits 112 receives the fluid sanitizer 60 from the primary conduit 108. Each of the secondary conduits 112 extends beneath a respective one of the pads 50. The sprayer 104 includes a plurality of nozzles 114 that is each fluidly coupled to a respective one of the secondary conduits 112 such that the plurality of nozzles 114 defines the plurality of spraying elements 106. Each of the nozzles 114 has a distal end 116 with respect to the respective secondary conduit 112 and the distal end 116 of each of the nozzles 114 is directed toward a respective one of the pads 50. The distal end 116 of each of the nozzles 114 is open thereby facilitating the distal end 116 of each of the nozzles 114 to spray the fluid disinfectant upwardly through the respective pad 50.

In use, the canister 90 is placed in the housing 62, the cover 94 is attached to the housing 62 and the floor mat 12 is positioned at a desired location, which may be an entrance to a home. The user 16 stands on each of the pads 50 when the user 16 enters the home. The pressure switch 56 is turned on when the user 16 stands on each of the pads 50 and the pump 84 urges the sanitizing fluid outwardly through the nozzles 114. In this way the sanitizing fluid is distributed onto the sole 20 of each shoe 22 to inhibit the shoe 22 from tracking bacteria into the home. The canister 90 is replaced with the canister 90 is depleted of the sanitizing fluid.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A shoe sanitizing assembly for sanitizing soles of shoes while the shoes are being worn, said assembly comprising:
a floor mat being positionable on a floor wherein said floor mat is configured to be stood upon by a user, said floor mat having a pair of foot areas being defined on said floor mat wherein each of said foot areas is configured to have a sole of a respective shoe being placed in said foot areas, each of said foot areas being comprised of a fluid permeable material;
a pressure switch being integrated into said floor mat wherein said pressure switch is configured to be compressed when the user stands on said floor mat, said pressure switch being turned on when the user stands on said floor mat;
a reservoir unit being coupled to said floor mat, said reservoir unit being aligned with a respective one of four corners of said floor mat, said reservoir unit containing a fluid sanitizer, said reservoir unit being in operational communication with said pressure switch, said reservoir unit being actuated to release a pre-determined volume of said fluid sanitizer when said pressure switch is turned on; and
a sprayer being integrated into said floor mat, said sprayer being in fluid communication with said reservoir unit such that said sprayer receives said fluid sanitizer from said reservoir unit, said sprayer having a plurality of spraying elements each being aligned with a respective one of said foot areas in said floor mat wherein each of said spraying elements is configured to spray said fluid sanitizer on the soles of the shoes when the user steps on said floor mat.

2. The assembly according to claim 1, wherein said floor mat comprises:
a pan having a bottom wall and a perimeter wall extending upwardly from said bottom wall, said pan being comprised of a rigid material, said pan being comprised of a fluid impermeable material; and
a panel having a bottom surface, a top surface and a perimeter surface extending between said top surface and said bottom surface, said perimeter surface being bonded to said perimeter wall of said pan such that said panel completely fills an area defined by said perimeter wall of said pan, said bottom surface being spaced from an upper surface of said bottom wall of said pan.

3. The assembly according to claim 2, wherein said perimeter surface of said panel has a front side, a back side, a first lateral side and a second lateral side, said panel having a pair of holes each extending through said top surface and said bottom surface such that each of said holes defines a respective one of said foot areas, each of said holes being elongated along an axis extending between said front side and said back side of said perimeter surface such that each of said holes has an ovoid shape, each of said holes having a bounding edge, said bounding edge of each of said holes undulating along opposing sides of said ovoid shape defined by said holes such that each of said holes has a shape that approximates the shape of the sole of the shoe, each of said holes being positioned on opposite sides of a center of said panel.

4. The assembly according to claim 3, wherein said floor mat includes a pair of pads, each of said pads having an outer edge, each of said pads being positioned in a respective one of said holes having said outer edge abutting said bounding edge of said respective hole such that each of said pads completely fills said respective hole wherein each of said pads is configured to be stood upon, each of said pads being comprised of a fluid permeable material, an upper surface of each of said pads being aligned with said top surface of said panel.

5. The assembly according to claim 4, wherein said pressure switch is positioned between said bottom wall of said pan and said panel, said panel pressing downwardly on said pressure switch when the user stands on said panel, said pressure switch being elongated to extend between each of said pads.

6. The assembly according to claim 2, wherein:
said reservoir unit comprises a housing having a lower disk, an upper disk and a riser extending between said lower disk and said upper disk, said lower disk having an outer surface and a topmost surface, said upper disk having a lowermost surface, said riser having a lateral edge;
said lateral edge having an engagement being disposed on said lateral edge;
said outer surface of said lower disk being coupled to said perimeter wall of said pan of said floor mat; and
said upper disk having a power supply compartment being disposed on said lowermost surface, said power supply compartment having a set of contacts being integrated into said power supply compartment, each of said set of contacts being comprised of an electrically conductive material.

7. The assembly according to claim 6, wherein said reservoir unit includes a pump being disposed on said topmost surface of said lower disk, said pump having an intake and an exhaust, said pump being electrically coupled to said pressure switch, said pump being turned on when said pressure switch is turned on, said pump being electrically coupled to said pressure switch.

8. The assembly according to claim 7, wherein said reservoir unit includes a canister containing said fluid sanitizer, said canister having an outlet, said canister being removably positionable in said housing having said outlet fluidly engaging said intake of said pump, said pump urging a pre-determined volume of said fluid sanitizer outwardly from said canister when said pump is turned on.

9. The assembly according to claim 6, wherein said reservoir unit includes a cover having a first lateral edge and a second lateral edge, said cover being concavely arcuate between said first lateral edge and said second lateral edge, said first lateral edge having an engaging element being disposed on said first lateral edge, said cover being positionable in said housing having said cover extending between said upper disk and said lower disk, said engaging element releasably engaging said engagement on said lateral edge of said riser when said cover is positioned in said housing.

10. The assembly according to claim 6, wherein said reservoir unit includes a power supply being insertable into said power supply compartment, said power supply being placed in electrical communication with each of set of contacts in said power supply compartment when said power supply is inserted into said power supply compartment.

11. The assembly according to claim 1, wherein:
said floor mat comprises:
a pan having a bottom wall and a perimeter wall extending upwardly from said bottom wall, said pan being comprised of a rigid material, said pan being comprised of a fluid impermeable material; and
a panel having a bottom surface, a top surface and a perimeter surface extending between said top surface and said bottom surface, said perimeter surface being bonded to said perimeter wall of said pan such that said panel completely fills an area defined by said perimeter wall of said pan, said bottom surface being spaced from an upper surface of said bottom wall of said pan;
said reservoir unit includes a pump having an exhaust; and
said sprayer comprises a primary conduit being positioned between said bottom surface of said panel and said upper surface of said bottom wall of said pan, said primary conduit having a first end being fluidly coupled to said exhaust of said pump such that said primary conduit receives said fluid sanitizer from said pump.

12. The assembly according to claim 11, wherein said sprayer includes a pair of secondary conduits each being fluidly coupled to said primary conduit such that each of said secondary conduits receives said fluid sanitizer from said primary conduit, each of said secondary conduits extending beneath a respective one of said pads.

13. The assembly according to claim 12, wherein said sprayer includes a plurality of nozzles, each of said nozzles being fluidly coupled to a respective one of said secondary conduits such that said plurality of nozzles defines said plurality of spraying elements, each of said nozzles having a distal end with respect to said respective secondary conduit, said distal end of each of said nozzles being directed toward a respective one of said pads, said distal end of each of said nozzles being open thereby facilitating said distal end of each of said nozzles to spray said fluid disinfectant upwardly through said respective pad.

14. A shoe sanitizing assembly for sanitizing soles of shoes while the shoes are being worn, said assembly comprising:
a floor mat being positionable on a floor wherein said floor mat is configured to be stood upon by a user, said floor mat having a pair of foot areas being defined on said floor mat wherein each of said foot areas is configured to have a sole of a respective shoe being placed in said foot areas, each of said foot areas being comprised of a fluid permeable material, said floor mat comprising:
a pan having a bottom wall and a perimeter wall extending upwardly from said bottom wall, said pan being comprised of a rigid material, said pan being comprised of a fluid impermeable material;
a panel having a bottom surface, a top surface and a perimeter surface extending between said top surface and said bottom surface, said perimeter surface being bonded to said perimeter wall of said pan such that said panel completely fills an area defined by said perimeter wall of said pan, said bottom surface being spaced from an upper surface of said bottom wall of said pan, said perimeter surface having a front side, a back side, a first lateral side and a second lateral side, said panel having a pair of holes each extending through said top surface and said bottom surface such that each of said holes defines a respective one of said foot areas, each of said holes being elongated along an axis extending between said front side and said back side of said perimeter surface such that each of said holes has an ovoid shape, each of said holes having a bounding edge, said bounding edge of each of said holes undulating along opposing sides of said ovoid shape defined by said holes such that each of said holes has a shape that approximates the shape of the sole of the shoe, each of said holes being positioned on opposite sides of a center of said panel; and
a pair of pads, each of said pads having an outer edge, each of said pads being positioned in a respective one of said holes having said outer edge abutting said bounding edge of said respective hole such that each of said pads completely fills said respective hole wherein each of said pads is configured to be stood upon, each of said pads being comprised of a fluid permeable material, an upper surface of each of said pads being aligned with said top surface of said panel;
a pressure switch being integrated into said floor mat wherein said pressure switch is configured to be compressed when the user stands on said floor mat, said pressure switch being turned on when the user stands on said floor mat, said pressure switch being positioned between said bottom wall of said pan and said panel, said panel pressing downwardly on said pressure switch when the user stands on said panel, said pressure switch being elongated to extend between each of said pads;
a reservoir unit being coupled to said floor mat, said reservoir unit being aligned with a respective one of four corners of said floor mat, said reservoir unit containing a fluid sanitizer, said reservoir unit being in operational communication with said pressure switch, said reservoir unit being actuated to release a pre-determined volume of said fluid sanitizer when said pressure switch is turned on, said reservoir unit comprising:
a housing having a lower disk, an upper disk and a riser extending between said lower disk and said upper disk, said lower disk having an outer surface and a topmost surface, said upper disk having a lowermost surface, said riser having a lateral edge, said lateral edge having an engagement being disposed on said lateral edge, said outer surface of said lower disk being coupled to said perimeter wall of said pan of said floor mat, said upper disk having a power supply compartment being disposed on said lowermost surface, said power supply compartment having a set of contacts being integrated into said power supply compartment, each of said set of contacts being comprised of an electrically conductive material;
a pump being disposed on said topmost surface of said lower disk, said pump having an intake and an exhaust, said pump being electrically coupled to said pressure switch, said pump being turned on when said pressure switch is turned on, said pump being electrically coupled to said pressure switch;
a canister containing said fluid sanitizer, said canister having an outlet, said canister being removably positionable in said housing having said outlet fluidly engaging said intake of said pump, said pump urging a pre-determined volume of said fluid sanitizer outwardly from said canister when said pump is turned on;
a cover having a first lateral edge and a second lateral edge, said cover being concavely arcuate between said first lateral edge and said second lateral edge, said first lateral edge having an engaging element being disposed on said first lateral edge, said cover being positionable in said housing having said cover extending between said upper disk and said lower disk, said engaging element releasably engaging said engagement on said lateral edge of said riser when said cover is positioned in said housing; and
a power supply being insertable into said power supply compartment, said power supply being placed in electrical communication with each of set of contacts in said power supply compartment when said power supply is inserted into said power supply compartment; and
a sprayer being integrated into said floor mat, said sprayer being in fluid communication with said reservoir unit such that said sprayer receives said fluid sanitizer from said reservoir unit, said sprayer having a plurality of spraying elements each being aligned with a respective one of said foot areas in said floor mat wherein each of said spraying elements is configured to spray said fluid sanitizer on the soles of the shoes when the user steps on said floor mat, said sprayer comprising:
a primary conduit being positioned between said bottom surface of said panel and said upper surface of said bottom wall of said pan, said primary conduit having first end being fluidly coupled to said exhaust of said pump such that said primary conduit receives said fluid sanitizer from said pump;
a pair of secondary conduits each being fluidly coupled to said primary conduit such that each of said secondary conduits receives said fluid sanitizer from said primary conduit, each of said secondary conduits extending beneath a respective one of said pads; and
a plurality of nozzles, each of said nozzles being fluidly coupled to a respective one of said secondary conduits such that said plurality of nozzles defines said plurality of spraying elements, each of said nozzles having a distal end with respect to said respective secondary conduit, said distal end of each of said nozzles being directed toward a respective one of said pads, said distal end of each of said nozzles being open thereby facilitating said distal end of each of said nozzles to spray said fluid disinfectant upwardly through said respective pad.
